(19) United States
(12) Patent Application Publication
(10) Pub. No.: US 2025/0361562 A1
(43) Pub. Date: Nov. 27, 2025

(54) CELL-FREE DNA SIGNALS AS BIOMARKERS OF PREECLAMPSIA

(71) Applicant: ILLUMINA, INC., San Diego, CA (US)

(72) Inventors: Sucheta Dhananjay Bhatt, San Diego, CA (US); Theresa Ann Boomer, San Diego, CA (US); Jean Gekas, Quebec (CA); Sung Kim, San Diego, CA (US); Weida Gong, San Ramon, CA (US); Christina Ngoc Bich Nguyen, San Diego, CA (US)

(73) Assignee: ILLUMINA, INC., San Diego, CA (US)

(21) Appl. No.: 18/874,202

(22) PCT Filed: Feb. 16, 2024
(86) PCT No.: PCT/US2024/016093

§ 371 Date: Dec. 12, 2024

Related U.S. Application Data
(60) Provisional application No. 63/446,404, filed on Feb. 17, 2023.
(60) Provisional application No. 63/552,234, filed on Feb. 12, 2024.

(51) Int. Cl. C12Q 1/6883; C12Q 1/6874

(52) U.S. Cl. CPC C12Q 1/6883; C12Q 1/6874; C12Q 2600/112; C12Q 2600/118

(57) ABSTRACT

The present invention includes methods and computer programs for use in the detection preeclampsia and/or determining an increased risk for preeclampsia in a pregnant female, the methods including identifying in a biosample obtained from the pregnant female cell-free DNA signals, including concentration, fetal fraction, and fragment size distribution. These methods provide for the identification of patients at risk of preeclampsia in the first trimester of pregnancy.

### CONTINUING APPLICATION DATA

This application claims the benefit of U.S. Provisional Application Ser. No. 63/446,404, filed Feb. 17, 2023, and U.S. Provisional Application Ser. No. 63/552,234, filed Feb. 12, 2024, each of which is incorporated by reference herein.

### FIELD OF INVENTION

The present invention relates generally to methods and materials for use in the detection and early risk assessment for the pregnancy complication preeclampsia.

### BACKGROUND

Preeclampsia is a condition that occurs only during pregnancy, affecting 5% to 8% of all pregnancies. It is the direct cause of 10%-15% of maternal deaths and 40% of fetal deaths. The three main symptoms of preeclampsia may include high blood pressure, swelling of hands and feet, and excess protein in the urine (proteinuria), occurring after week 20 of pregnancy. Other signs and symptoms of preeclampsia may include severe headaches, changes in vision (including temporary loss of vision, blurred vision, or light sensitivity), nausea or vomiting, decreased urine output, decreased platelets levels (thrombocytopenia), impaired liver function, and shortness of breath, caused by fluid in the lung. See, for example Steegers et al., 2010, Lancet; 376:631-644. doi: 10.1016/S0140-6736(10)60279-6; and Miller et al., 2008, Semin Perinatol; 32:274-280. doi: 10.1053/j.semperi.2008.04.010.

The more severe the preeclampsia and the earlier it occurs in pregnancy, the greater the risks for mother and baby. Preeclampsia may require induced labor and delivery or delivery by cesarean delivery. Left untreated, preeclampsia can lead to serious, even fatal, complications for both the mother and baby. Complications of preeclampsia include fetal growth restriction FGR), low birth weight, preterm birth, placental abruption, HELLP syndrome (hemolysis, elevated liver enzymes, and low platelet count syndrome), eclampsia (a severe form of preeclampsia that leads to seizures), organ damage, including kidney, liver, lung, heart, eye damage, stroke, or other brain injury. See, for example, “Preeclampsia-Symptoms and causes-Mayo Clinic,” Apr. 15, 2022, available at on the worldwide web at mayoclinic.org/diseases-conditions/preeclampsia/symptoms-causes/syc-20355745.

With early detection and treatment, most women can deliver a healthy baby if preeclampsia is detected early and treated with regular prenatal care. Although various protein biomarkers display changed levels in maternal serum at presymptomatic stages, these biomarkers lack discriminative and predictive power in individual patients (Karumanchi and Granger, 2016, Hypertension; 67(2): 238-242). Thus, the identification of biomarkers for the early detection of preeclampsia is critical for the early diagnosis and treatment of preeclampsia.

### SUMMARY OF THE INVENTION

In one aspect, this disclosure describes a method of detecting preeclampsia and/or determining an increased risk for preeclampsia in a pregnant female subject, the method comprising:
providing cell free DNA (cfDNA) sequence information obtained from a biosample obtained from the pregnant female subject; and
from the cfDNA sequence information:
   determining cell free DNA (cfDNA) concentration;
   determining fetal fraction within the cfDNA; and/or
   determining fragment size distribution within the cfDNA;
      wherein:
a higher cfDNA concentration relative to a normal control;
a lower fetal fraction within the cfDNA relative to a normal control;
a higher fragment size distribution relative to a normal control; and/or
the ratio of fetal fraction to fragment size distribution is less than this ratio in a normal control
   is indicative of preeclampsia and/or an increased risk for preeclampsia in the pregnant female.

In some aspects of the methods disclosed herein, the cfDNA sequence information is obtained from sequencing based non-invasive prenatal testing (NIPT) testing.

In one aspect, this disclosure describes a method of detecting preeclampsia and/or determining an increased risk for preeclampsia in a pregnant female subject, the method comprising:
removing intact cells from a biosample obtained from the pregnant female;
isolating cell free DNA (cfDNA) molecules from the biosample;
sequencing the resulting enriched cfDNA to obtain cfDNA sequence information; and
from the cfDNA sequence information:
   determining cell free DNA (cfDNA) concentration;
   determining fetal fraction within the cfDNA; and/or
   determining fragment size distribution within the cfDNA;
      wherein:
a higher cfDNA concentration relative to a normal control;
a lower fetal fraction within the cfDNA relative to a normal control;
a higher fragment size distribution relative to a normal control; and/or
the ratio of fetal fraction to fragment size distribution is less than this ratio in a normal control
   is indicative of preeclampsia and/or an increased risk for preeclampsia in the pregnant female.

In some aspects of the methods disclosed herein, the method comprises:
determining fetal fraction within the cfDNA; and
determining fragment size distribution within the cfDNA;
   wherein:
a lower fetal fraction within the cfDNA relative to a normal control; and
a higher fragment size distribution relative to a normal control;
   is indicative of preeclampsia and/or an increased risk for preeclampsia in the pregnant female.

In some aspects of the methods disclosed herein, the method comprises:
determining cell free DNA (cfDNA) concentration;
determining fetal fraction within the cfDNA; and
determining fragment size distribution within the cfDNA;
   wherein:
a higher cfDNA concentration relative to a normal control;
a lower fetal fraction within the cfDNA relative to a normal control; and
a higher fragment size distribution relative to a normal control;
   is indicative of preeclampsia and/or an increased risk for preeclampsia in the pregnant female.

In one aspect, this disclosure describes a method comprising:
a) accessing sequence reads from cfDNA and determining:
   i) cfDNA concentration;
   ii) fetal fraction within the cfDNA;
   iii) fragment size distribution within the cfDNA; and/or
   iv) a parameter comprising the fetal fraction and the fragment size distribution;
b) comparing
   i) the cfDNA concentration to a first threshold amount;
   ii) the fetal fraction to a second threshold amount;
   iii) the fragment size distribution to a third threshold amount; and/or
   iv) the parameter comprising the fetal fraction and the fragment size distribution to a fourth threshold amount;
wherein the first, second, third and fourth threshold amounts are determined based on a plurality of normal samples;
c) determining that the pregnant female subject has preeclampsia and/or an increased risk for preeclampsia, wherein
   i) the cfDNA concentration is higher than the first threshold amount;
   ii) the fetal fraction is lower than the second threshold amount;
   iii) the fragment size distribution is higher than the third threshold amount; and/or
   iv) the parameter comprising the fetal fraction and the fragment size distribution is less than the fourth threshold amount;
      is indicative of preeclampsia and/or an increased risk for preeclampsia in the pregnant female.

In some aspects of the methods disclosed herein, the biosample is obtained from the pregnant female at less than 16 weeks gestation.

In some aspects of the methods disclosed herein, the biosample is obtained from the pregnant female subject at about 11 to about 14.2 weeks gestation.

In some aspects of the methods disclosed herein, the biosample is obtained from the pregnant female at greater than 20 weeks gestation.

In some aspects of the methods disclosed herein, the biosample is obtained from the pregnant female subject at about 17.6 to about 25.5 weeks gestation.

In some aspects of the methods disclosed herein, detecting preeclampsia and/or determining an increased risk for preeclampsia comprises detecting early-onset preeclampsia and/or determining an increased risk for early-onset preeclampsia.

In some aspects of the methods disclosed herein, detecting preeclampsia and/or determining an increased risk for preeclampsia comprises detecting late onset preeclampsia and/or determining an increased risk for late onset preeclampsia.

In some aspects of the methods disclosed herein, the biosample comprises whole blood, serum, or plasma.

In some aspects of the methods disclosed herein, the method further comprises providing the pregnant female with a therapeutic intervention selected from the group consisting of increased frequency of prenatal visits, antihypertensive medications to lower blood pressure, corticosteroid medications, anticonvulsant medications, bed rest, hospitalization, early delivery, and combinations thereof, and/or treating the pregnant female with a low dose of aspirin, wherein a low dose of aspirin comprises about 50 to about 150 mg per day.

In one aspect, this disclosure describes a system comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform a method as described herein.

In one aspect, this disclosure describes a machine comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform a method as described herein.

In one aspect, this disclosure describes a non-transitory computer-readable storage medium with an executable program stored thereon, where the program instructs a microprocessor to access sequence reads mapped to a reference genome and perform a method as described herein.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (for example, found in deoxyribonucleic acid (DNA)) or a ribose sugar (for example, found in ribonucleic acid (RNA)). A nucleic acid can contain any of a variety of analogs of these sugar moieties that are known in the art. A nucleic acid can include native or nonnative bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine. Useful nonnative bases that can be included in a nucleic acid are known in the art. The term “template” and “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

As used herein, “amplify,” “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the target nucleic acid molecule. The target nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

As used herein, “amplification conditions” and its derivatives, generally refers to conditions suitable for amplifying one or more nucleic acid sequences. Such amplification can be linear or exponential. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences, or to amplify an amplified target sequence ligated to one or more adapters, e.g., an adapter-ligated amplified target sequence. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending, and separating are repeated. Typically, the amplification conditions include cations such as Mg⁺⁺ or Mn⁺⁺ and can also include various modifiers of ionic strength.

As used herein, the term “polymerase chain reaction” (PCR) refers to the method of U.S. Pat. Nos. 4,683,195 and 4,683,202, which describes a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double-stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

As used herein, the term “primer” and its derivatives refer generally to any polynucleotide that can hybridize to a target sequence of interest. Typically, the primer functions as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some embodiments, the primer is a single-stranded oligonucleotide or polynucleotide. The terms “polynucleotide” and “oligonucleotide” are used interchangeably herein to refer to a polymeric form of nucleotides of any length, and may comprise ribonucleotides, deoxyribonucleotides, analogs thereof, or mixtures thereof. The terms should be understood to include, as equivalents, analogs of either DNA or RNA made from nucleotide analogs and to be applicable to single stranded (such as sense or antisense) and double-stranded polynucleotides. The term as used herein also encompasses cDNA, that is complementary or copy DNA produced from an RNA template, for example by the action of reverse transcriptase. This term refers only to the primary structure of the molecule. Thus, the term includes triple-, double- and single-stranded deoxyribonucleic acid (“DNA”), as well as triple-, double- and single-stranded ribonucleic acid (“RNA”).

As used herein, the terms “library” and “sequencing library” refer to a collection or plurality of template molecules which share common sequences at their 5′ ends and common sequences at their 3′ ends. The collection of template molecules containing known common sequences at their 3′ and 5′ ends may also be referred to as a 3′ and 5′ modified library.

The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “sensitivity” as used herein is equal to the number of true positives divided by the sum of true positives and false negatives.

The term “specificity” as used herein is equal to the number of true negatives divided by the sum of true negatives and false positives.

The term “enrich” herein refers to the process of amplifying nucleic acids contained in a portion of a sample. Enrichment includes specific enrichment that targets specific sequences, e.g., polymorphic sequences, and non-specific enrichment that amplifies the whole genome of the DNA fragments of the sample.

The term “maternal sample” herein refers to a biological sample obtained from a pregnant subject, e.g., a woman.

The term “biological fluid” herein refers to a liquid taken from a biological source and includes, for example, blood, serum, plasma, sputum, lavage fluid, cerebrospinal fluid, urine, semen, sweat, tears, saliva, and the like. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

The terms “maternal nucleic acids” and “fetal nucleic acids” herein refer to the nucleic acids of a pregnant female subject and the nucleic acids of the fetus being carried by the pregnant female, respectively.

As used herein, the term “fetal fraction” refers to the fraction of fetal nucleic acids present in a sample comprising fetal and maternal nucleic acid. Fetal fraction is often used to characterize the cfDNA in a mother's blood.

As used herein, the term “each,” when used in reference to a collection of items, is intended to identify an individual item in the collection but does not necessarily refer to every item in the collection unless the context clearly dictates otherwise.

As used herein, “providing” in the context of a composition, an article, a nucleic acid, or a nucleus means making the composition, article, nucleic acid, or nucleus, purchasing the composition, article, nucleic acid, or nucleus, or otherwise obtaining the compound, composition, article, or nucleus.

The term “and/or” means one or all of the listed elements or a combination of any two or more of the listed elements. The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1A-1D. Use of cfDNA signals to distinguish between late PE, early PE, and control samples at timepoint A. Use of concentration at timepoint A to distinguish between the three patient cohorts (FIG. 1A); Use of fragment size distribution at timepoint A to distinguish between the three patient cohorts (FIG. 1B); Use of fetal fraction at timepoint A to distinguish between the three patient cohorts (FIG. 1C); Linear regression plot showing the use of fetal fraction and fragment size distribution to distinguish between PE samples and control samples at timepoint A (FIG. 1D). The Asterix (*) denotes significance between that particular PE group and the control group.

FIG. 2A-2D. Use of cfDNA signals to distinguish between late PE, early PE, and control samples at timepoint B. Use of concentration at timepoint B to distinguish between the three patient cohorts (FIG. 2A); Use of fragment size distribution at timepoint B to distinguish between the three patient cohorts (FIG. 2B); Use of fetal fraction at timepoint B to distinguish between the three patient cohorts (FIG. 2C); Linear regression plot showing the use of fetal fraction and fragment size distribution to distinguish between PE samples and control samples at timepoint B (FIG. 2D). The Asterix (*) denotes significance between that particular PE group and the control group.

FIGS. 3A and 3B. FIG. 3A is a graph of fetal fraction-fragment size distribution-concentration at time point A. FIG. 3B is a graph of fetal fraction-fragment size distribution-concentration at time point B.

FIG. 4 is a linear regression plot showing the use of fetal fraction and fragment size distribution to distinguish between PE samples and control samples at timepoint A. PE samples are denoted by square. Control samples are denoted by squares.

FIG. 5 is a plot of probability versus outcome.

The schematic drawings are not necessarily to scale. Like numbers used in the figures may refer to like components. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

The present disclosure determined that cell-free DNA (cfDNA) can be used as an efficient biomarker for the identification of patients at risk of developing preeclampsia (PE). Using a logistic regression model, the three cfDNA signals of concentration, fetal fraction, and fragment size distribution were correlated with clinical outcomes. For example, significant differences were observed between PE patients and control cases for all three cfDNA signals at timepoint A (about 11 to about 14.2 weeks gestation), while both fetal fraction and concentration were significantly different between PE patients and control cases at timepoint B (about 17.6 to about 25.5 weeks gestation). Overall, the model had a sensitivity of up to 100% and specificity of up to 87.5% at timepoint A. The use of this logistic regression model can identify patients at risk in the first trimester of pregnancy.

The present disclosure includes methods of detecting preeclampsia and/or determining an increased risk for preeclampsia in a pregnant female subject, the method including providing cfDNA sequence information obtained from a biosample obtained from a pregnant female subject and determining cfDNA concentration, determining fetal fraction within the cfDNA, and/or determining fragment size distribution from the cfDNA sequence information.

With a method as disclosed herein a higher cfDNA concentration relative to a normal control, a lower fetal fraction within the cfDNA relative to a normal control, a higher fragment size distribution relative to a normal control, and/or a ratio of fetal fraction to fragment size distribution that is less than this ratio in a normal control is indicative of preeclampsia and/or an increased risk for preeclampsia in the pregnant female.

In some embodiments, two of these three cfDNA signals may be measured and may be indicative of preeclampsia and/or an increased risk for preeclampsia in the pregnant female. For example, cfDNA concentration and fetal fraction may be determined, wherein a higher cfDNA concentration relative to a normal control and a lower fetal fraction within the cfDNA relative to a normal control is indicative of preeclampsia and/or an increased risk for preeclampsia in the pregnant female. In some embodiments, cfDNA concentration and fragment size distribution may be determined, wherein a higher cfDNA concentration relative to a normal control and a higher fragment size distribution relative to a normal control is indicative of preeclampsia and/or an increased risk for preeclampsia in the pregnant female. Or, fetal fraction and fragment size distribution may be determined, wherein a lower fetal fraction within the cfDNA relative to a normal control and a higher fragment size distribution relative to a normal control is indicative of preeclampsia and/or an increased risk for preeclampsia in the pregnant female.

In some embodiments, all three cfDNA signals may be determined and may be indicative of preeclampsia and/or an increased risk for preeclampsia in the pregnant female. For example, cfDNA concentration, fetal fraction, and fragment size distribution are determined, wherein a higher cfDNA concentration relative to a normal control, a lower fetal fraction within the cfDNA relative to a normal control, and a higher fragment size distribution relative to a normal control is indicative of preeclampsia and/or an increased risk for preeclampsia in the pregnant female.

In come embodiments, a ratio of two of the three cfDNA signals may be indicative of preeclampsia and/or an increased risk for preeclampsia in the pregnant female. For example, a ratio of fetal fraction to fragment size distribution that is less than this ratio in a normal control is indicative of preeclampsia and/or an increased risk for preeclampsia in the pregnant female. In a further embodiment, the ratio of cfDNA concentration to fetal fraction relative to this ratio in a normal control may be indicative of preeclampsia and/or an increased risk for preeclampsia in the pregnant female. In a further embodiment, the ratio of cfDNA concentration to fragment size distribution relative to this ratio in a normal control may be indicative of preeclampsia and/or an increased risk for preeclampsia in the pregnant female.

The concentration of cell-free DNA (cfDNA) of fetal origin circulating in maternal plasma, referred to as fetal fraction (FF). Fetal fraction concentration is the percentage of total cell-free DNA (cfDNA) in a sample derived from the fetus.

In some implementations, the one or more values of fetal fraction include a value of fetal fraction calculated using the information about the sizes of the cell-free nucleic acid fragments. In some implementations, the value of fetal fraction is calculated by obtaining a frequency distribution of the fragment sizes; and applying the frequency distribution to a model relating fetal fraction to frequency of fragment size to obtain the fetal fraction value. In some implementations, the model relating fetal fraction to frequency of fragment size includes a general linear model having a plurality of terms and coefficients for a plurality of fragment sizes.

In some implementations, the one or more values of fetal fraction include a value of fetal fraction calculated using coverage information for the bins of the reference genome. In some implementations, the value of fetal fraction is calculated by applying coverage values of a plurality of bins to a model relating fetal fraction to coverage of bins to obtain the fetal fraction value. In some implementations, the model relating fetal fraction to the coverage of bin includes a general linear model having a plurality of terms and coefficients for a plurality of bins. In some implementations, the plurality of bins have high correlation between fetal fraction and coverage in training samples. One example of such a method is described in U.S. Pat. No. 10,622,094, the content of which is incorporated by reference in its entirety.

In some implementations, the one or more values of fetal fraction include a value of fetal fraction calculated using frequencies of a plurality of 8-mers found in the reads. In some implementations, the value of fetal fraction is calculated by applying frequencies of a plurality of 8-mers to a model relating fetal fraction to 8-mer frequency to obtain the fetal fraction value. In some implementations, the model relating fetal fraction to 8-mer frequency includes a general linear model having a plurality of terms and coefficients for a plurality of 8-mers. In some implementations, the plurality of 8-mers have high correlation between fetal fraction and 8-mer frequency.

In some implementations, the one or more values of fetal fraction include a value of fetal fraction calculated using coverage information for the bins of a sex chromosome.

In certain embodiments, the amount of fetal nucleic acid is determined according to markers specific to a male fetus (e.g., Y-chromosome STR markers (e.g., DYS 19, DYS 385, DYS 392 markers); RhD marker in RhD-negative females), allelic ratios of polymorphic sequences, or according to one or more markers specific to fetal nucleic acid and not maternal nucleic acid (e.g., differential epigenetic biomarkers (e.g., methylation; described in further detail below) between mother and fetus, or fetal RNA markers in maternal blood plasma.

Determination of fetal nucleic acid content (e.g., fetal fraction) sometimes is performed using a fetal quantifier assay (FQA). This type of assay allows for the detection and quantification of fetal nucleic acid in a maternal sample based on the methylation status of the nucleic acid in the sample. In certain embodiments, the amount of fetal nucleic acid from a maternal sample can be determined relative to the total amount of nucleic acid present, thereby providing the percentage of fetal nucleic acid in the sample. Methods for differentiating nucleic acid based on methylation status include, but are not limited to, methylation sensitive capture, for example, using a MBD2-Fc fragment in which the methyl binding domain of MBD2 is fused to the Fc fragment of an antibody (MBD-FC); methylation specific antibodies; bisulfite conversion methods, for example, MSP (methylation-sensitive PCR), COBRA, methylation-sensitive single nucleotide primer extension (Ms-SNuPE) or Sequenom MassCLEAVE™ technology; and the use of methylation sensitive restriction enzymes (e.g., digestion of maternal DNA in a maternal sample using one or more methylation sensitive restriction enzymes thereby enriching the fetal DNA). Methyl-sensitive enzymes also can be used to differentiate nucleic acid based on methylation status, which, for example, can preferentially or substantially cleave or digest at their DNA recognition sequence if the latter is non-methylated. Thus, an unmethylated DNA sample will be cut into smaller fragments than a methylated DNA sample and a hypermethylated DNA sample will not be cleaved.

In certain embodiments, fetal fraction can be determined based on allelic ratios of polymorphic sequences (e.g., single nucleotide polymorphisms (SNPs). In such a method, nucleotide sequence reads are obtained for a maternal sample and fetal fraction is determined by comparing the total number of nucleotide sequence reads that map to a first allele and the total number of nucleotide sequence reads that map to a second allele at an informative polymorphic site (e.g., SNP) in a reference genome. In certain embodiments, fetal alleles are identified, for example, by their relative minor contribution to the mixture of fetal and maternal nucleic acids in the sample when compared to the major contribution to the mixture by the maternal nucleic acids. Accordingly, the relative abundance of fetal nucleic acid in a maternal sample can be determined as a parameter of the total number of unique sequence reads mapped to a target nucleic acid sequence on a reference genome for each of the two alleles of a polymorphic site.

The distribution of different-sized fragments in a sample of cfDNA can be measured and compared to the fragment size distribution of a reference sample. One way to measure fragment size distribution utilizes a cumulative distribution function. The fragment size distribution of a test sample can be compared to a reference (or expected) distribution using any one of a number of approaches known to those of skill in the art. In some embodiments, a Kolmogorov-Smirnov test (K-S test) can be applied to compare the sample distribution with a reference probability distribution. In some embodiments, an autocorrelation can be applied to compare the sample distribution with a reference probability distribution. In some embodiments, a cross correlation can be applied to compare the sample distribution with a reference probability distribution. For example, in embodiments presented herein, “frag_size_dist” is a metric generated during analysis of cfDNA and is defined as the standard deviation of the difference between actual and expected cumulative fragment size distributions.

An appropriate control may be a normal control, for example, a sample from a healthy, gestational age-matched pregnant female. In some embodiments, the results of an appropriate control have been previously obtained and are recorded and stored as historical results, on for example, an electronic storage medium.

While most of the DNA and RNA in the body is located within cells, extracellular nucleic acids can also be found circulating freely in the blood. Cell-free DNA (cfDNA) refers to non-encapsulated DNA in the blood stream. Cell-free DNA are short fragments of DNA released into the bloodstream through a natural process of cell death. It was first discovered by Mandel and Métais in 1948 (Mandel and Metais, 1948, C R Seances Soc Biol Fil; 142:241-243). cfDNA can be found in plasma and other body fluids.

Previous studies have indicated that most of the plasma cfDNA molecules originate from the hematopoietic system in healthy individuals (Lui et al., 2002, Clin Chem; 48:421-427; and Sun et al., 2015, Proc Natl Acad Sci USA; 112:E5503-E5512). However, in certain physiological or pathological conditions, such as pregnancy, organ transplantation, and cancers, the related/affected tissues release additional DNA into peripheral circulation (Leon et al., 1977, Cancer Res; 37:646-650; Lo et al., 1997, Lancet; 350:485-487; and Lo et al., 1998, Lancet, 351:1329-1330).

In recent years, a variety of technologies have emerged based on the analysis of cfDNA for noninvasive prenatal testing (NIPT) (Lo et al., 1997, Lancet; 350:485-487; Lo et al., 2007, Proc Natl Acad Sci USA; 104:13116-13121; Hyett et al., 2005, Prenat Diagn; 25:1111-1116; Wong and Lo, 2015, Trends Mol Med; 21:98-108; Hudecova and Chiu, 2017, Best Pract Res Clin Obstet Gynaecol; 39:63-73; Bianchi and Chiu, 2018, N Engl J Med, 379:464-473; Malan et al., 2018, JAMA; 320:557-565; Vivanti et al., 2019, Ultrasound Obstet Gynecol; 53:87-94; and Zhang J. et al., 2019, Nat Med; 25:439-447), for monitoring organ transplantation (Lo et al., 1998, Lancet, 351:1329-1330; Gielis et al., 2015, Am J Transplant, 15:2541-2551; Gala-Lopez et al., 2018, Transplantation; 102:978-985; and Sherwood and Weimer, 2018), and for detecting immune diseases (Zhang et al., 2014; Beranek et al., 2017, Arch Dermatol Res; 309:815-821; Dunaeva et al., 2018, Mol Neurobiol; 55:4681-4688; Xu et al., 2018, Eur J Clin Invest; 48:e13015; and Duvvuri and Lood, 2019, Front Immunol; 10:502) as well as cancers.

During pregnancy, the mother's blood contains cell-free DNA (cfDNA), both from her own tissue, and from the fetus via the placenta. Approximately 2-20% of total cfDNA in maternal blood is placental (Barrett et al., 2011, PLoS One; 6(10):e25202; and Nigam et al., 2012, J Interntl Med Sci Acad; 25(3):119-120).

Prenatal testing in recent years has been moving towards non-invasive methods to determine the fetal risk for genetic disorders. Noninvasive prenatal testing (NIPT) for fetal chromosome abnormalities using cf DNA in the maternal circulation became clinically available in the United States in October 2011. Assaying the cell-free fetal DNA present in maternal plasma via various molecular methods is now used to identify a range of fetal chromosomal aneuploidies, such as, for example, trisomy 21, trisomy 13, and trisomy 18, determine the sex of the fetus, and identify various gene mutations, such as, for example, Tay-Sachs disease, sickle cell anemia, thalassemia, cystic fibrosis, muscular dystrophy, and fragile X syndrome. Noninvasive prenatal testing for fetal chromosome abnormalities using cell-free DNA in the maternal circulation may be for screening purposes or for diagnostic purposes.

The detection, quantification, and/or characterization of cfDNA in maternal circulation associated with a diagnosis of preeclampsia or a risk for developing preeclampsia of the methods described herein may involve any of a variety of technologies, including, but not limited to, any of a variety of high-throughput sequencing techniques. For example, methods for assaying cfDNA includes, but is not limited to, and of the methods described herein or used in the examples provided herein.

In some embodiments, the detection, quantification, and/or characterization of cfDNA in maternal circulation associated with a diagnosis of preeclampsia or a risk for developing preeclampsia of the methods described may be undertaken as a part of a NIPT aneuploidy screening of a maternal sample. The detection, quantification, and/or characterization of cfDNA in maternal circulation associated with a diagnosis of preeclampsia or a risk for developing preeclampsia can be performed, for example, before, concurrent with, or after such NIPT analysis is performed. In certain embodiments presented here, detection of preeclampsia or a risk for developing preeclampsia makes use of certain metrics obtained during NIPT analysis. As an example, some metrics that can be determined in an NIPT analysis include total cfDNA concentration, fragment size, fragment size distribution, and fetal fraction. Since maternal plasma samples represent a mixture of maternal and fetal cfDNA, the success of any given NIPT method depends on its sensitivity to detect copy number changes in the low fetal fraction samples. For counting based methods, their sensitivity is determined by (a) sequencing depth and (b) ability of data normalization to reduce technical variance. The analytical methodology for NIPT and other applications can include deriving fragment size information from, e.g., paired-end reads, and using this information in an analysis pipeline. Improved analytical sensitivity provides the ability to apply NIPT methods at reduced coverage (e.g., reduced sequencing depth) which enables the use of the technology for lower-cost testing of average risk pregnancies.

A typical NIPT analysis pipeline includes determining copy number and copy number variations (CNV) of different sequences of interest in a test sample that comprises a mixture of nucleic acids derived from two or more different genomes, and which are known or are suspected to differ in the amount of one or more sequence of interest. Copy number variations determined by such methods include gains or losses of entire chromosomes, alterations involving very large chromosomal segments that are microscopically visible, and an abundance of sub-microscopic copy number variation of DNA segments ranging from single nucleotides, to kilobases (kb), to megabases (Mb) in size.

Some implementations of NIPT analysis include fragment length (or fragment size) of cfDNA to improve sensitivity and specificity for fetal aneuploidy detection from cfDNA in maternal plasma. Some embodiments are implemented with a PCR free library preparation coupled with paired end DNA sequencing. In some embodiments, both fragment size and coverage are utilized to enhance fetal aneuploidy detection. In some embodiments, the methods involve combining independent counting of shorter fragments with the relative fraction of shorter fragments in bins across the genome.

Some implementations of NIPT analysis include methods to improve the sensitivity and/or specificity of sequence data analysis by removing within-sample GC-content bias. In some embodiments, removal of within-sample GC-content bias is based on sequence data corrected for systematic variation common across unaffected training samples.

Some implementations of NIPT analysis include methods to derive parameters with high signal to noise ratio from cell free nucleic acid fragments, for determining various genetic conditions related to copy number and CNV, with improved sensitivity, selectivity, and/or efficiency relative to conventional methods. The parameters include, but are not limited to, coverage, fragment size weighted coverage, fraction, or ratio of fragments in a defined range, methylation level of fragments, t-statistics obtained from coverage, fetal fraction estimates obtained from coverage information, etc. The depicted process has been found particularly effective at improving the signal in samples having relatively low fractions of DNA from a genome under consideration (e.g., a genome of a fetus). An example of such sample is a maternal blood sample from an individual pregnant with fraternal twins, triplets, etc., where the process assesses copy number variation in the genome of one of the fetuses.

In some implementations of NIPT analysis, high analytical sensitivities and specificities can be achieved with a simple library preparation using very low cfDNA input that does not require PCR amplification. The PCR free method simplifies the workflow, improves the turnaround time, and eliminates biases that are inherent with PCR methods. In some embodiments, the detection of fetal aneuploidy from maternal plasma can be made more robust and efficient than conventional methods, requiring fewer unique cfDNA fragments. In combination, improved analytical sensitivity and specificity is achieved with a very fast turnaround time at a significantly lower number of cfDNA fragments. This potentially allows NIPT to be carried out at significantly lower costs to facilitate application in the general obstetric population.

In various implementations of NIPT analysis, PCR-free library preparation is possible with the disclosed methods. Some implementations eliminate inherent biases of PCR methods, reduced assay complexity, reduce required sequencing depth (2.5× lower), provide faster turnaround time, e.g., turn around in one day, enable in-process fetal fraction (FF) measurement, facilitate discrimination between maternal and fetal/placental cfDNA using fragment size information.

In some embodiments, the detection, quantification, and/or characterization of cfDNA in maternal circulation associated with a diagnosis of preeclampsia or a risk for developing preeclampsia is performed as part of a NIPT protocol using methods such as those described in U.S. Pat. No. 10,095,831, the content of which is incorporated by reference herein in its entirety. In some embodiments, the detection, quantification, and/or characterization of cfDNA in maternal circulation associated with a diagnosis of preeclampsia or a risk for developing preeclampsia is performed as part of a NIPT protocol using methods such as Illumina's VERISEQ™ NIPT Solution v2 assay (Illumina, Inc., Foster City, CA).

A sample may be a biological sample or biosample, including but not limited to blood, serum, plasma, sweat, tears, urine, sputum, lymph, saliva, amniotic fluid, a tissue biopsy, swab, or smear, including for example, but not limited to, a placental tissue sample. In some preferred embodiments, a biological sample is a cell free plasma sample. A biological sample may be a maternal sample obtained from a pregnant female subject.

As used herein, the term “subject” refers to a human subject as well as a non-human mammalian subject. Although the examples herein concern humans and the language is primarily directed to human concerns, the concept of this disclosure is applicable to any mammal, and is useful in the fields of veterinary medicine, animal sciences, research laboratories and such.

A subject may be a pregnant female, including a pregnant female in any gestational stages of pregnancy. The gestational stage of pregnancy may be, for example, the first trimester, the second trimester, including late second trimester, or the third trimester, including early third trimester.

In some embodiments, the gestational stage of pregnancy may be, for example, before about 10 weeks of pregnancy, before about 11 weeks of pregnancy, before about 12 weeks of pregnancy, before about 16 weeks of pregnancy, before about 20 weeks of pregnancy, or before about 25 weeks of pregnancy.

In some embodiments, the gestational stage of pregnancy may be, for example, about 10 weeks gestation, about 11 weeks gestation, about 12 weeks gestation, about 13 weeks gestation, about 14 weeks gestation, about 15 weeks gestation, about 16 weeks gestation, about 17 weeks gestation, about 18 weeks gestation, about 19 weeks gestation, about 20 weeks gestations, about 21 weeks gestation, about 22 weeks gestation, about 23 weeks gestation, about 24 weeks gestation, about 25 weeks gestation, about 26 weeks gestation, about 27 weeks gestation, about 28 weeks gestation, about 29 weeks gestation, about 30 weeks gestation, about 31 weeks gestation, about 32 weeks gestation, about 33 weeks gestation, about 34 weeks gestation, about 35 weeks gestation, about 36 weeks gestation, or any range thereof. For example, in some embodiments, the gestational stage of pregnancy may be, for example, about 8 to about 18 weeks of gestation, about 9 to about 12 weeks gestation, about 9 to about 14 weeks gestation, about 10 to about 14 weeks of gestation, about 10 to about 15 weeks of gestation, about 11 to about 14 weeks of gestation, about 11 to about 13 weeks of gestation, about 11 to about 15 weeks of gestation, about 12 to about 13 weeks of gestation, about 12 to about 14 weeks of gestation, about 12 to about 28 weeks of gestation, about 13 to about 16 weeks gestation, about 17 to about 20 weeks gestation, about 17 to about 25 weeks gestation, about 17 to about 26 weeks gestation, about 18 to about 26 weeks gestation, about 21 to about 24 weeks gestation, about 24 to about 27 weeks gestation, or about 25 to about 28 weeks gestation.

In some embodiments, the gestational stage of pregnancy may be, for example, about 11 to about 14.2 weeks gestation or 17.6 to about 25.5 weeks gestation.

In some embodiments, the methods described herein can detect early onset preeclampsia and/or determine an increased risk for early onset preeclampsia, wherein early onset preeclampsia is the onset of preeclampsia before about 34 weeks of gestation.

In some embodiments, the methods described herein can detect late onset preeclampsia and/or determine an increased risk for late onset preeclampsia, wherein late onset preeclampsia is the onset of preeclampsia at or after about 34 weeks of gestation.

The disclosure includes kits for use in the diagnosis of preeclampsia and the identification of pregnant women at risk for developing preeclampsia. A kit is any manufacture (for example, a package or container) including at least one reagent for specifically detecting, quantifying, and/or characterizing the cfDNA signals of concentration, fetal fraction, and/or fragment size distribution within the maternal circulation as described herein that is indicative of preeclampsia or a risk for developing preeclampsia. The kit may be promoted, distributed, or sold as a unit for performing the methods of the present disclosure.

The use of cell free DNA biomarkers found in the maternal circulation indicative of preeclampsia in noninvasive methods for the diagnosis of preeclampsia and the identification of pregnant women at risk for developing preeclampsia may be combined with appropriate monitoring and medical management. For example, further tests may be ordered. Such test may include, for example, blood tests to measure liver function, kidney function, and/or platelet and various clotting proteins, urine analysis to measure protein or creatinine levels, fetal ultrasound to measure monitor fetal growth, weight, and amniotic fluid, a nonstress test to measure how fetal heart rate with fetal movement, and/or a biophysical profile using ultrasound to measure your fetal breathing, muscle tone, and movement and the volume of amniotic fluid may be ordered. Therapeutic interventions may include, for example, increasing the frequency of prenatal visits, antihypertensive medications to lower blood pressure, corticosteroid medications, anticonvulsant medications, bed rest, hospitalization, and/or early delivery. See, for example, Townsend et al., 2016 “Current best practice in the management of hypertensive disorders in pregnancy,” Integr Blood Press Control; 9: 79-94.

Therapeutic interventions may include the administration of low dose aspirin to pregnant women identified at risk of for developing preeclampsia. A recent multicenter, double-blind, placebo-controlled trial demonstrated that treatment of women at high risk for preterm preeclampsia with low-dose aspirin resulted in a lower incidence of this diagnosis compared to placebo (Rolnik et al., 2017, N Engl J Med; 377(7):613-622). Meta-analyses of randomized trials found that an appropriate daily dose of aspirin initiated before 16 weeks of gestation could prevent most cases of PE as well as its related complications (Bujold, 2022, Hypertension; 79:323-324; Bujold et al., 2010, Obstet Gynecol; 116:402-414; and Roberge et al., 2018, Am J Obstet Gynecol; 218(3):287-293e1). The American College of Obstetricians and Gynecologists (ACOG) recommends that women with any of the high-risk factors for PE and those with more than one moderate risk factor should receive low-dose aspirin starting between 12 and 28 weeks of gestation (preferably before 16 weeks' gestation) until delivery (ACOG Practice Bulletin, Number 222. Gestational Hypertension and Preeclampsia. Obstetrics and gynecology 2020, 135, e237-e260, doi:10.1097/aog.0000000000003891). It is therefore optimal that screening for PE is carried out in the first trimester of pregnancy.

Dosages of low dose aspirin include, but are not limited to, about 50 to about 150 mg per day, about 60 to about 80 mg per day, about 100 or more mg per day, or about 150 mg per day. Administration may begin, for example, at or before 16 weeks of gestation or from 11 to 14 weeks of gestation. Administration may continue thru 36 weeks of gestation.

The present disclosure also includes a system comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform a method as described herein.

The present disclosure also includes a machine comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform a method as described herein.

The present disclosure also includes a non-transitory computer-readable storage medium with an executable program stored thereon, where the program instructs a microprocessor to access sequence reads mapped to a reference genome and perform a method as described herein.

### EXEMPLARY ASPECTS

The invention is defined in the claims. However, below there is provided a non-exhaustive listing of non-limiting exemplary aspects. Any one or more of the features of these aspects may be combined with any one or more features of another example, embodiment, or aspect described herein. Exemplary Embodiments of the present invention include, but are not limited to, the following.

Aspect 1 includes a method of detecting preeclampsia and/or determining an increased risk for preeclampsia in a pregnant female subject, the method comprising:
providing cell free DNA (cfDNA) sequence information obtained from a biosample obtained from the pregnant female subject; and
from the cfDNA sequence information:
   determining cell free DNA (cfDNA) concentration;
   determining fetal fraction within the cfDNA; and/or
   determining fragment size distribution within the cfDNA;
      wherein:
a higher cfDNA concentration relative to a normal control;
a lower fetal fraction within the cfDNA relative to a normal control;
a higher fragment size distribution relative to a normal control; and/or
the ratio of fetal fraction to fragment size distribution is less than this ratio in a normal control
   is indicative of preeclampsia and/or an increased risk for preeclampsia in the pregnant female.

Aspect 2 is the method of aspect 1, wherein the cfDNA sequence information is obtained from sequencing based non-invasive prenatal testing (NIPT) testing.

Aspect 3 is a method of detecting preeclampsia and/or determining an increased risk for preeclampsia in a pregnant female subject, the method comprising:
removing intact cells from a biosample obtained from the pregnant female;
isolating cell free DNA (cfDNA) molecules from the biosample;
sequencing the resulting enriched cfDNA to obtain cfDNA sequence information; and
from the cfDNA sequence information:
   determining cell free DNA (cfDNA) concentration;
   determining fetal fraction within the cfDNA; and/or
   determining fragment size distribution within the cfDNA;
      wherein:
a higher cfDNA concentration relative to a normal control;
a lower fetal fraction within the cfDNA relative to a normal control;
a higher fragment size distribution relative to a normal control; and/or
the ratio of fetal fraction to fragment size distribution is less than this ratio in a normal control
   is indicative of preeclampsia and/or an increased risk for preeclampsia in the pregnant female.

Aspect 4 is the method of any one of aspects 1 to 3, comprising:
determining fetal fraction within the cfDNA; and
determining fragment size distribution within the cfDNA;
   wherein:
a lower fetal fraction within the cfDNA relative to a normal control; and
a higher fragment size distribution relative to a normal control;
   is indicative of preeclampsia and/or an increased risk for preeclampsia in the pregnant female.

Aspect 5 is the method of any one of aspects 1 to 4, comprising:
determining cell free DNA (cfDNA) concentration;
determining fetal fraction within the cfDNA; and
determining fragment size distribution within the cfDNA;
   wherein:
a higher cfDNA concentration relative to a normal control;
a lower fetal fraction within the cfDNA relative to a normal control; and
a higher fragment size distribution relative to a normal control;
   is indicative of preeclampsia and/or an increased risk for preeclampsia in the pregnant female.

Aspect 6 is a method comprising:
a) accessing sequence reads from cfDNA and determining:
   i) cfDNA concentration;
   ii) fetal fraction within the cfDNA;
   iii) fragment size distribution within the cfDNA; and/or
   iv) a parameter comprising the fetal fraction and the fragment size distribution;
b) comparing
   i) the cfDNA concentration to a first threshold amount;
   ii) the fetal fraction to a second threshold amount;
   iii) the fragment size distribution to a third threshold amount; and/or
   iv) the parameter comprising the fetal fraction and the fragment size distribution to a fourth threshold amount;
wherein the first, second, third and fourth threshold amounts are determined based on a plurality of normal samples;
c) determining that the pregnant female subject has preeclampsia and/or an increased risk for preeclampsia, wherein
   i) the cfDNA concentration is higher than the first threshold amount;
   ii) the fetal fraction is lower than the second threshold amount;
   iii) the fragment size distribution is higher than the third threshold amount; and/or
   iv) the parameter comprising the fetal fraction and the fragment size distribution is less than the fourth threshold amount;
      is indicative of preeclampsia and/or an increased risk for preeclampsia in the pregnant female.

Aspect 7 is the method of any one of aspects 1 to 6, wherein the biosample is obtained from the pregnant female at less than 16 weeks gestation.

Aspect 8 is the method of any one of aspects 1 to 7, wherein the biosample is obtained from the pregnant female subject at about 11 to about 14.2 weeks gestation.

Aspect 9 is the method of any one of aspects 1 to 6, wherein the biosample is obtained from the pregnant female at greater than 20 weeks gestation.

Aspect 10 is the method of any one of aspect s 1 to 6, wherein the biosample is obtained from the pregnant female subject at about 17.6 to about 25.5 weeks gestation.

Aspect 11 is the method of any one of aspects 1 to 10, wherein detecting preeclampsia and/or determining an increased risk for preeclampsia comprises detecting early-onset preeclampsia and/or determining an increased risk for early-onset preeclampsia.

Aspect 12 is the method of any one of aspects 1 to 10, wherein detecting preeclampsia and/or determining an increased risk for preeclampsia comprises detecting late onset preeclampsia and/or determining an increased risk for late onset preeclampsia.

Aspect 13 is the method of any one of aspects 1 to 12, wherein the biosample comprises whole blood, serum, or plasma.

Aspect 14 is the method of any one of aspects 1 to 13, further comprising providing the pregnant female with a therapeutic intervention selected from the group consisting of increased frequency of prenatal visits, antihypertensive medications to lower blood pressure, corticosteroid medications, anticonvulsant medications, bed rest, hospitalization, early delivery, and combinations thereof, and/or treating the pregnant female with a low dose of aspirin, wherein a low dose of aspirin comprises about 50 to about 150 mg per day.

Aspect 15 is a system comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform the method of any one of aspects 1 to 14.

Aspect 16 is a machine comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform the method of any one of aspects 1 to 14.

Aspect 17 is a non-transitory computer-readable storage medium with an executable program stored thereon, where the program instructs a microprocessor to access sequence reads mapped to a reference genome and perform the method of any one of aspects 1 to 14.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Use of Cell-Free DNA Signals as Biomarkers for Early and Easy Prediction of Preeclampsia

Preeclampsia (PE) is a leading cause of maternal and perinatal morbidity worldwide. However, current methods of screening are complicated and require special skill sets. With this example, cell-free (cf) DNA as an efficient biomarker for the identification of at-risk patients was evaluated. One hundred patients attending a private prenatal clinic in Canada were enrolled in their first trimester of pregnancy and a blood draw was carried out at 11.0-14.2 weeks' (timepoint A) and 17.6-25.5 weeks of gestation (timepoint B). Twelve patients developed PE—four early-stage and eight late-stage PE. CfDNA signals, namely concentration, fetal fraction, and fragment size distribution, were correlated with clinical outcomes in the test population to develop a logistic regression model. Significant differences were observed between PE patients and control cases for all three cfDNA signals at timepoint A, while both fetal fraction and concentration were significantly different between PE patients and control cases at timepoint B. Overall, this model had a sensitivity of up to 100% and specificity of up to 87.5% at timepoint A. This study showed that use of this logistic regression model could identify patients at risk of preeclampsia in the first trimester of pregnancy.

### Introduction

Worldwide, preeclampsia (PE) typically affects 2%-8% of pregnant women and is one of the leading causes of maternal and perinatal morbidity (Steegers et al., 2010, Lancet; 376(9741):631-644). Severe PE can lead to preterm birth, fetal growth restriction, maternal multiorgan dysfunction, maternal seizures, and perinatal death, with 76,000 women and 500,000 babies dying from this disorder every year (Poon et al., 2019, Int J Gynaecol Obstet; 145(Suppl 1): 1-33; and Bujold, 2022, Hypertension; 79:323-324). In addition, mothers that had PE and children from affected pregnancies have an increased risk of long-term cardiovascular disease and chronic diseases, with the life expectancy of women who developed preterm PE being reduced on average by 10 years (Poon et al., 2019, Int J Gynaecol Obstet; 145(Suppl 1):1-33; and Chaemsaithong et al., 2022, Am J Obstet Gynecol; 226(2S):S1071-S1097.e2). Preeclampsia is a multifaceted syndrome with a highly variable clinical presentation and can be classified into two main sub-types, namely early-onset PE and late-onset PE. The International Society for the Study of Hypertension in Pregnancy (ISSHP) classify (de novo) PE as gestational hypertension accompanied by one of three new-onset conditions at ≥20 weeks of gestation, namely proteinuria, other maternal end organ dysfunction, or uteroplacental dysfunction (Magee et al., 2022, Pregnancy Hypertens; 27:148-169).

There are a number of risk factors associated with development of PE including advanced maternal age, chronic hypertension, autoimmune diseases such as antiphospholipid antibody syndrome and systemic lupus erythematosus, pregestational diabetes, and multifetal pregnancies (Poon et al., 2019, Int J Gynaecol Obstet; 145(Suppl 1):1-33; and Burton et al., 2019, BMJ; 366:12381). Currently, there is no specific treatment for PE, with premature induction of labor being the only treatment for severe cases. Metaanalyses of randomized trials found that an appropriate daily dose of aspirin initiated before 16 weeks of gestation could prevent most cases of PE as well as its related complications (Bujold, 2022, Hypertension; 79:323-324; Bujold et al., 2010, Obstet Gynecol; 116:402-414; and Roberge et al., 2018, Am J Obstet Gynecol; 218(3):287-293e1). The American College of Obstetricians and Gynecologists (ACOG) recommends that women with any of the high risk factors for PE and those with more than one moderate risk factor should receive low-dose aspirin starting between 12 and 28 weeks of gestation (preferably before 16 weeks' gestation) until delivery (ACOG Practice Bulletin, Number 222. Gestational Hypertension and Preeclampsia. Obstetrics and gynecology 2020, 135, e237-e260, doi:10.1097/aog.0000000000003891). It is therefore optimal that screening for PE is carried out in the first trimester of pregnancy.

There have been a variety of different methods proposed for screening patients for PE during pregnancy. One of the main screening methods currently used is based on the Bayes based competing risk (CR) model. This PE screening method combines maternal characteristics and obstetric history with biomarkers for risk assessment including mean arterial pressure, uterine artery pulsatility index, and placental growth factor (PlGF) (Poon et al., 2009, Hypertension; 53:812-818; and Wright et al., 2015, Am J Obstet Gynecol; 213:62). Although this method is superior to the traditional approach that is based solely on maternal medical history and demographic characteristics, it is complicated and requires multiple different tests and information for calculating PE risk assessment.

This example determined that cell-free (cf) DNA can be used as an efficient biomarker for the identification of patients at risk of developing PE. Currently, cfDNA obtained from a maternal blood draw during pregnancy is used extensively for prenatal aneuploidy screening. This example looked at whether cfDNA-based signals at two different timepoints (11.0-14.2 weeks' gestation and 17.6-25.5 weeks' gestation) differed between patients that did and did not develop preeclampsia during their pregnancy. It also compared the screening performance of a proposed model to that of the CR model. This example found that the proposed model was able to distinguish between PE and control cases using cfDNA signals from the first trimester of pregnancy in a logistic regression model with a sensitivity of up to 100% and specificity of up to 87.5%.

### Materials and Methods

The test population for this proposed model consisted of samples from pregnant patients attending a private prenatal clinic in Quebec City (Cliniques Prenato, Canada) for first-trimester routine noninvasive prenatal testing (NIPT) for aneuploidy and other adverse obstetrical outcomes. Visits were held at 11.0-to 25.5 weeks of gestation from July 2020 to May 2021, where nurses took a record of maternal characteristics and medical history including maternal age; gestational age; maternal height and weight; racial origin; if the patient was a cigarette smoker during pregnancy; if there was any PE history in the mother of the patient; and the conception method for the current pregnancy. This was then reviewed by a specialised doctor.

The patients also had a blood draw for PlGF serum levels, their mean arterial pressure measured, and measurement of the left and right uterine artery pulsatility index using a transabdominal colour Doppler ultrasonography. Patients were enrolled in the study if they were at least 18 years of age, had a singleton pregnancy, and a gestational age (GA) of 11.0-14.2 weeks. Patients were excluded from the study if they had chronic hypertension, were taking aspirin or any anticoagulant drug, were on immunosuppressors, or either currently or previously had cancer.

A blood sample was collected as part of routine NIPT screening process at 11.0-14.2 weeks of gestation (timepoint A) with a repeat blood draw at 17.6-25.5 weeks of gestation (timepoint B) for the purposes of this study. Blood samples from timepoint A were processed using VERIFI™ lab developed test, an adapted version of Illumina's VERISEQ™ NIPT Solution v2 assay (Illumina, Inc. clinical services laboratory, Foster City, CA). Blood samples from timepoint B were run on the RUO version. The NIPT results on rare autosomal trisomies or copy number variations from either timepoint A or timepoint B were not reported to the patient. Only the validated results for trisomy 21, trisomy 18, and trisomy 13 from timepoint A were returned to the patient. All women gave their written consent to participate and share the obtained data in the study. This project has received approval from Prenato Clinics Institutional Research Review Board (Approval Number 12302019-2).

Follow-up was carried out on all study participants until delivery. The neonatal examination report was collected if an adverse outcome was reported. Outcome in-formation included birth weight, GA at delivery, method of delivery, and if there were any delivery complications. Outcomes included PE or delivery with PE and other ad-verse pregnancy/birth outcomes; PE was defined according to the International Society for the Study of Hypertension in Pregnancy (Brown et al., 2001, Hypertens Pregnancy, 20 (1): IX-XIV).

CfDNA parameters from the NIPT bioinformatics analysis for timepoint A were used in the development of the model. A multiple logistic regression model was fit where preeclampsia outcome was a dependent binary variable with cfDNA fetal fraction and fragment size distribution (FragSizeDist), +/− concentration (Conc), as independent variables. Specifically, the logistic regression predicted the probability of preeclampsia based on FragSizeDist as an independent variable, along with interaction terms that include FF and Conc. Supervised 5-fold cross validation was performed on the model with a minimum number of PE cases in the training and test sets. Training of the selected model utilized ˜80% of patient data (n=75, 8 PE cases+67 non-PE cases) with ˜20% of patient data (n=20, 4 PE cases+16 non-PE cases) used in the test set. The model presented with 90% accuracy.

All bioinformatic statistical analyses were performed using RStudio software program v 2022.07.2 build 576 © 2009-2022.

### Results

A total of 100 samples from pregnant patients were prospectively collected for this example. Five samples did not have outcome data and so were excluded from the study. Twelve patients developed PE, of which four were early-stage (before 34 weeks of gestation) and eight were late-stage PE (after 34 weeks of gestation); no aneuploidies were detected in the group of patients that developed PE. Patient demographics for the test population are shown in Table 1. Patients were divided into four separate categories, depending on their observed PE outcome and their screen-positive status by the Competing Risk model or this example's cfDNA signals model. Patients that had any adverse pregnancy outcomes were excluded from the control group. Biomarker levels were measured for all study participants and included blood pressure, uterine artery pulsatility index, and PlGF. Details of these for each of the four groups are also shown in Table 1.

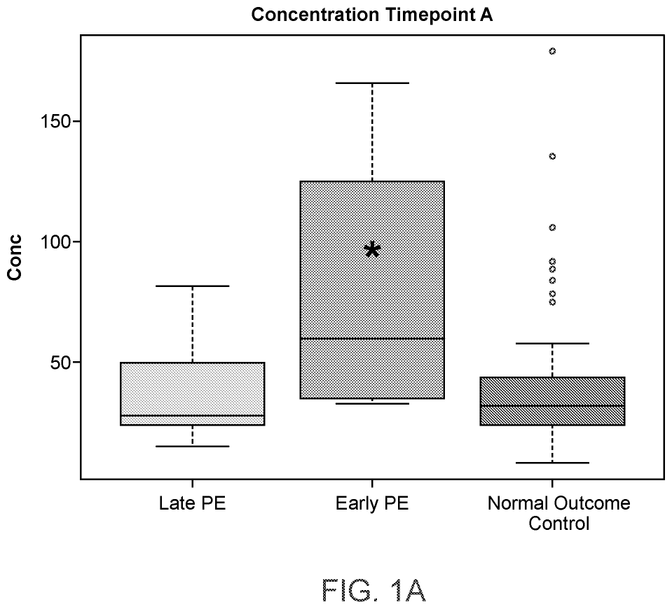

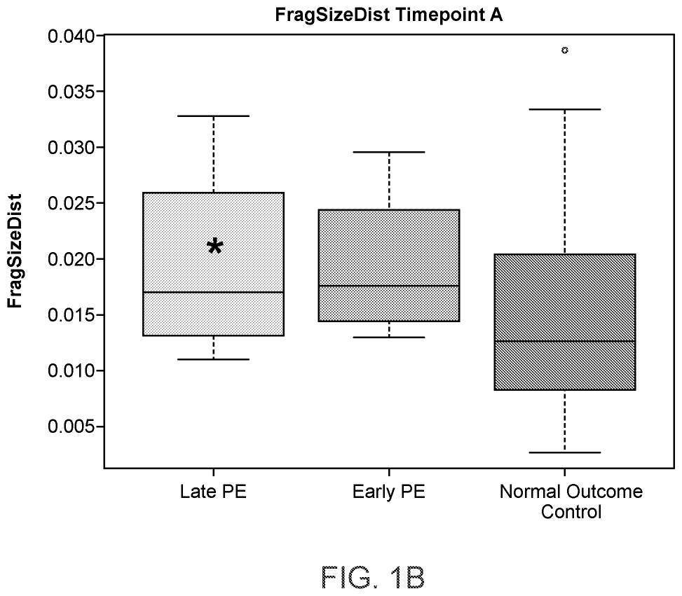

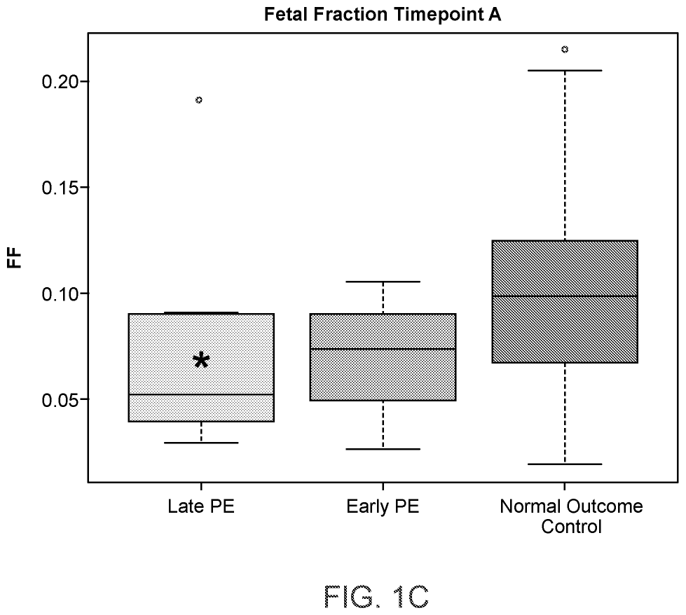

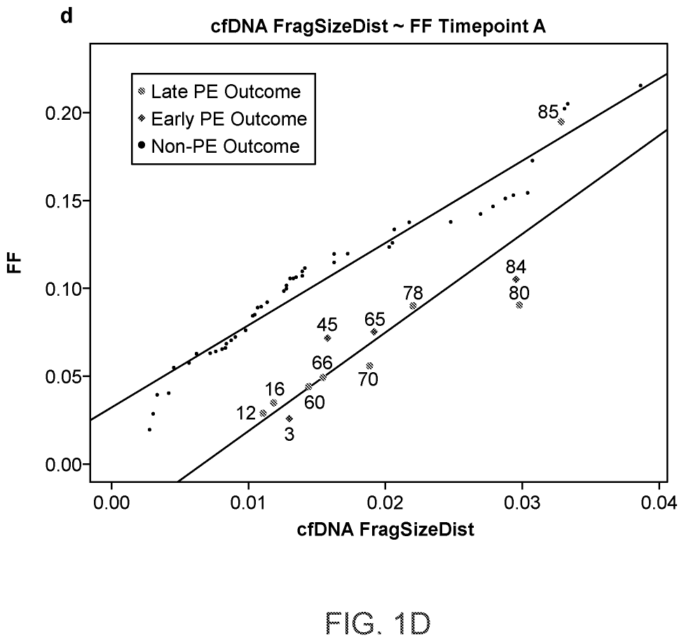

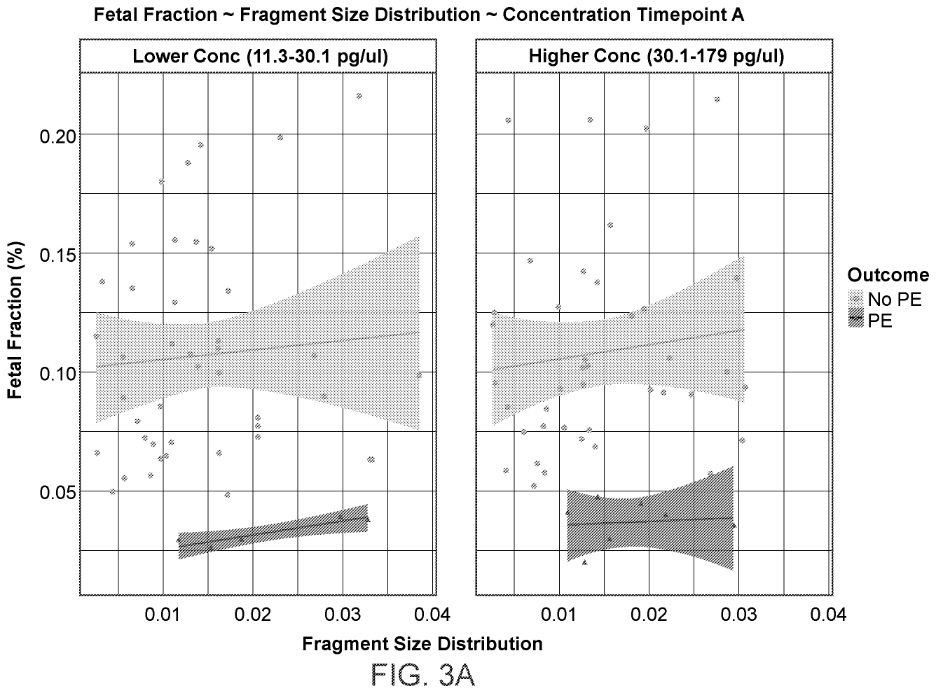

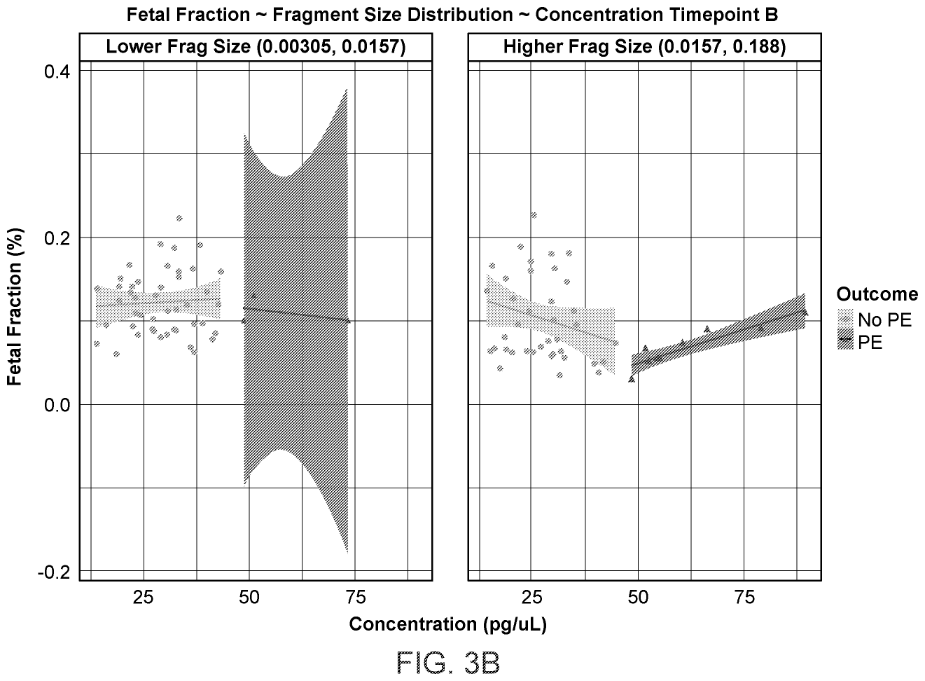

For the proposed model, three different cfDNA signals were considered, namely cfDNA concentration, fetal fraction, and fragment size distribution. FIGS. 1A-1C show the impact of these different cfDNA signals on distinguishing cases of early-stage and late-stage PE patients from control samples at timepoint A (11.0-14.2 weeks of gestation). As can be seen from FIG. 1A, the concentration of cfDNA from cases with early PE was significantly higher compared to the control samples (p=0.048), while no significant difference was observed between the late PE group and controls. Fragment size distribution was found to be significantly higher for the late-stage PE cases compared to controls (p=0.036; FIG. 1B), whilst fetal fraction was found to be significantly lower for the late-stage PE group compared to controls (p=0.028; FIG. 1C). In addition, linear regression plotting demonstrated that the use of fetal fraction and fragment size distribution could help distinguish between PE cases and control cases, apart from one outlier (case #85; FIG. 1D). A linear regression plot for timepoint A with all three cfDNA signals is shown in FIG. 3A.

TABLE 1
Maternal demograhics and pregnancy history of study participants.

| | Patients with no PE | | Patients with | | High-risk by CR | | High-risk by cfDNA | |
|---|---|---|---|---|---|---|---|---|
| | or APO | | observed PE | | model | | models* | |
| Variable | (n = 45) | | (n = 12) | | (n = 17) | | (n = 6) | |
| Maternal age, yr | 30.08 | (28.26-31.34) | 29.89 | (27.65-30.51) | 29.31 | (27.41-33.93) | 30.01 | (29.05-30.27) |
| Maternal height, cm | 166.50 | (166.00-170.00) | 163.00 | (155.00-165.00) | 163.00 | (155.00-165.00) | 164.00 | (157.00-165.00 |
| Maternal weight, kg | 67.30 | (60.23-76.78) | 79.45 | (62.15-92.51) | 73.40 | (61.70-82.40) | 78.80 | (74.98-104.98) |
| Body mass index, kg/m² | 24.51 | (22.32-27.33) | 27.59 | (25.84-37.39) | 27.56 | (23.56-33.24) | 32.91 | (28.45-39.88) |
| Gestational age, wk | 12.2 | (12.1-12.4) | 12.3 | (12.1-12.6) | 12.2 | (12.0-12.6) | 12.5 | (12.3-12.6) |
| Racial origin | | | | | | | | |
| White | 47 | (98) | 12 | (100) | 16 | (94) | 6 | (100) |
| Other | 1 | (2) | 0 | (0) | 1 | (6) | 0 | (0) |
| Cigarette smokers | 1 | (2) | 1 | (8) | 0 | (0) | 0 | (0) |
| Family history of PE | 2 | (4) | 0 | (0) | 2 | (12) | 0 | (0) |
| Method of conception | | | | | | | | |
| Natural | 47 | (98) | 11 | (92) | 15 | (88) | 6 | (100) |
| Ovulation induction | 0 | (0) | 1 | (8) | 2 | (12) | 0 | (0) |
| In vitro fertilization | 1 | (2) | 0 | (0) | 0 | (0) | 0 | (0) |
| Parity | | | | | | | | |
| Nulliparous | 22 | (46) | 11 | (92) | 15 | (88) | 4 | (67) |
| Parous, no previous PE | 26 | (54) | 1 | (8) | 2 | (12) | 2 | (33) |
| Parous, previous PE | 0 | (0) | 0 | (0) | 0 | (0) | 0 | (0) |
| Interpregnancy interval, yr | 1.80 | (1.30-2.10) | 0.30 | (0.30-0.30)** | 4.65 | (3.33-5.98) | 2.15 | (2.13-2.18) |
| Biomarker levels as measured | | | | | | | | |
| Systolic blood pressure (mmHg) | 105.88 | (101.69-113.31) | 120.00 | (113.38-127.25) | 118.25 | (115.50-123.75) | 121.38 | (106.81-126.19 |
| Diastolic blood pressure (mmHg) | 67.75 | (65.00-72.63) | 80.63 | (74.69-81.81) | 81.50 | (76.50-83.00) | 79.50 | (69.13-81.44) |
| Mean blood pressure (mmHg) | 81.13 | (77.73-85.17) | 93.46 | (88.85-97.17) | 93.92 | (89.25-98.23) | 93.46 | (81.77-96.27) |
| UAPI | 1.42 | (1.15-1.54) | 1.84 | (1.42-2.03) | 1.49 | (1.37-1.88) | 1.80 | (1.53-1.88) |
| PIGE (ng/ml) | 21.85 | (18.60-26.15) | 18.95 | (11.68-28.68) | 12.10 | (6.00-17.10) | 29.75 | (23.43-30.08) |
| Standardized biomarker levels | | | | | | | | |
| Mean blood pressure, MoM | 0.94 | (0.91-1.00) | 1.04 | (1.02-1.05) | 1.05 | (1.02-1.09) | 1.03 | (0.93-1.04) |
| UAPI MoM | 0.87 | (0.69-0.97) | 1.14 | (0.90-1.24) | 0.94 | (0.86-1.13) | 1.18 | (0.98-1.21) |
| PIGF MoM | 0.73 | (0.58-0.90) | 0.63 | (0.45-0.93) | 0.38 | (0.26-0.55) | 0.94 | (0.72-1.24) |

Data are expressed are median (interquartile range) or n (%) unless noted otherwise. APO, adverse pregnancy outcomes; MoM, Multiple of the median; PE, preeclampsia; PIGF, Placental growth factor; UAPI, Uterine artery pulsatility index; wk; week; yr, year.
*Based on the test set.
**Based on one patient only.

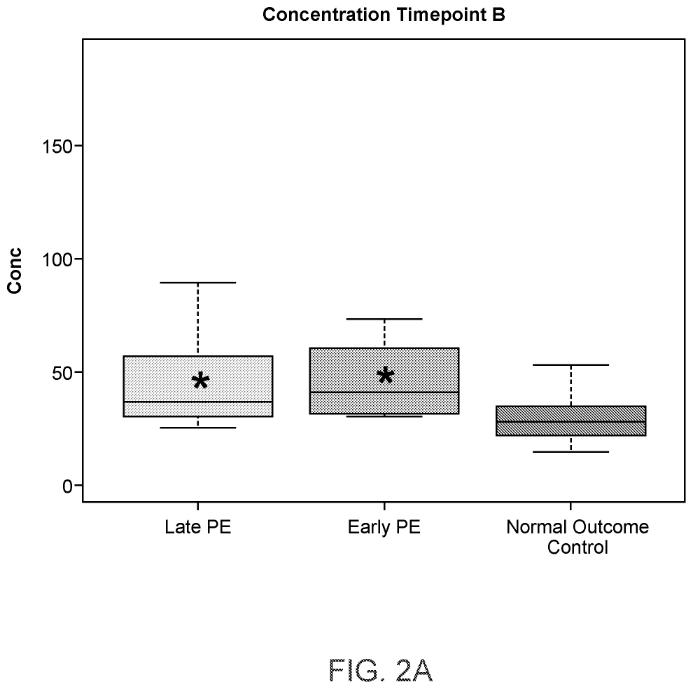

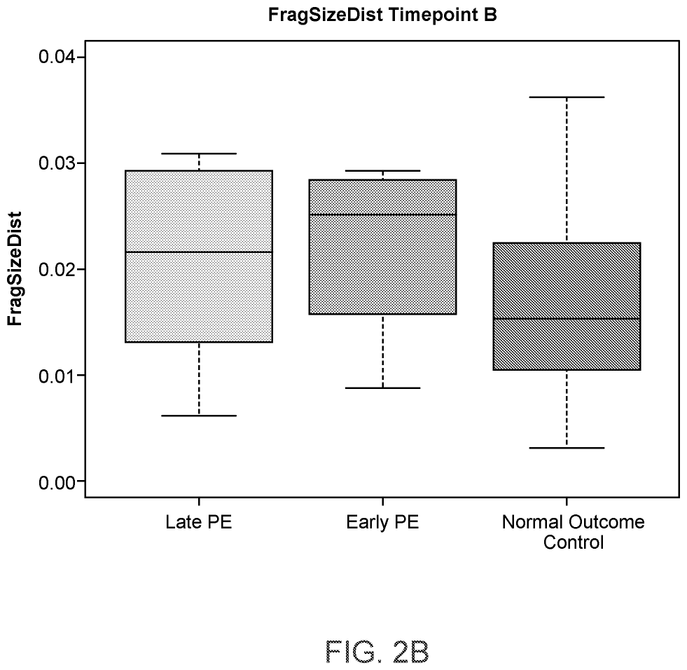

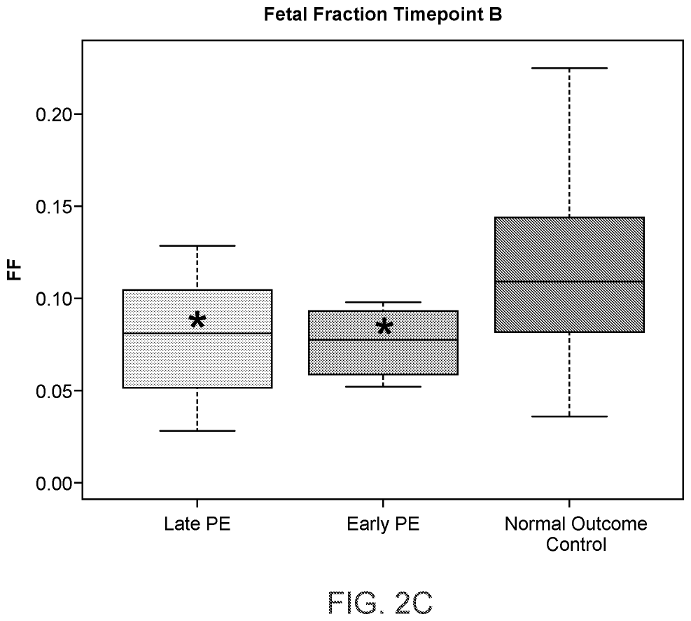

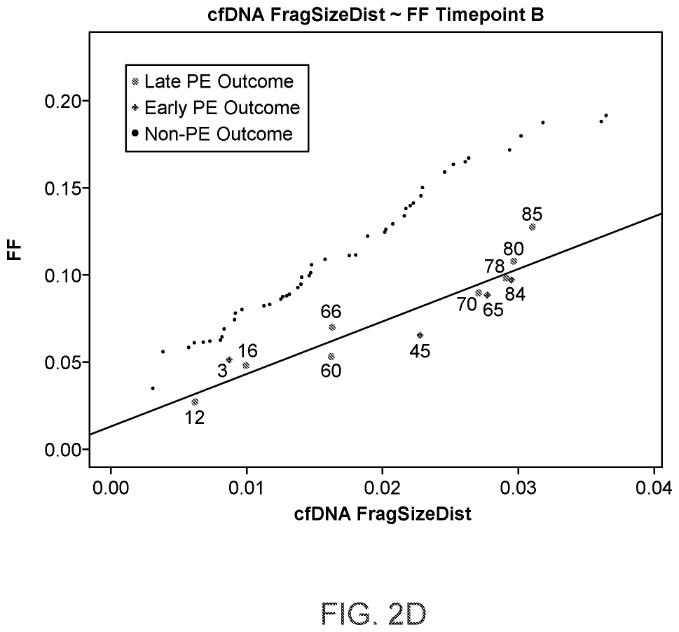

For timepoint B (17.6-25.5 weeks of gestation; FIGS. 2A-2C), cfDNA concentration was significantly higher for both the early-stage (p=0.021) and late-stage (p=0.007) PE cases compared to controls, whilst the FF was significantly lower for both the early-stage (p=0.044) and late-stage (p=0.015) PE cases compared to controls. No significant differences were observed for fragment size distribution at timepoint B. Similar to timepoint A, linear regression plotting demonstrated that the use of fetal fraction and fragment size distribution could help distinguish between PE cases and control cases (FIG. 2D). As can be seen, the outlier case (#85) from timepoint A aligned with the PE cases in timepoint B. A linear regression plot for timepoint A with all three cfDNA signals is shown in FIG. 3B.

As a final step, the performance of the proposed model (based on the test set of data) at both timepoint A and timepoint B with compared with the established CR model. As can be seen from Table 2, at timepoint A (first trimester of pregnancy), the instant model had a higher sensitivity (100% vs 58.3%) and higher PPV (66.7% vs 41.0%) compared to the CR model, with the same specificity observed for both models (87.5%).

TABLE 2
Summary of test performance for the cfDNA Screening Approach (n
= 20) and the Competing Risk Approach (n = 95).

| | Sensitivity, % | | Specificity, % | | False | False | PPV, % |
|---|---|---|---|---|---|---|---|
| Screening test for PE | (95% CI) | | (95% CI) | | positives, n | negatives, n | (95% CI) |
| cfDNA Approach (timepoint A) | 100 | (40-100) | 87.5 | (62.0-98.0) | 2 | 0 | 66.7 (22-96) |
| cfDNA Approach (timepoint B) | 100 | (40-100) | 75.0 | (48-93) | 4 | 0 | 50.0 (16-84) |
| Competing Risk Approach | 58.3 | (28.0-85.0) | 87.5 | (79.0-94.0) | 10 | 5 | 41.0 |

* CI, Confidence Interval; PPV, positive predictive value.

### Discussion

This example establishes that cfDNA-based signals can be used to differentiate between patients that develop PE during pregnancy and those that do not. The use of cfDNA signals in a logistic regression model was able to identify at risk patients, with a significant difference observed in concentration, fetal fraction, and fragment size distribution between PE patients and control cases.

As presented in this example, cfDNA concentration was significantly higher for early-stage PE patients at timepoint A and significantly higher for both early-stage and late-stage PE patients at timepoint B, compared to control cases. Similar to this example, a number of other studies have also noted an association between cfDNA concentration and preeclampsia. Lo et al. first demonstrated back in 1999 that the median concentration of circulating fetal DNA was much higher in preeclamptic patients than non-preeclamptic patients (Lo et al., 1999, Clin Chem; 45(2):184-188). The authors suggested that measurement of circulating DNA could possibly be a useful marker for diagnosis of PE. A more recent study found that total cfDNA was notably higher in preeclampsia at diagnosis, and that this higher total cfDNA correlated with an earlier gestational age at delivery and higher systolic blood pressure (Kolarova et al., 2021, J Am Heart Assoc; 10(15):e021477). Another study also found that PE was associated with higher maternal serum total cfDNA concentration than normal pregnancy (Rafaeli-Yehudai et al., 2018, PloS One; 13(7):e0200360). While a study by Rolnik et al. showed that there was a significant increase in median total cfDNA in early PE patients compared to controls, the authors found that measurements of total cfDNA at 11-13 weeks and 20-24 weeks of gestation were not predictive of PE (Rolnik et al., 2015, Ultrasound Obstet Gynecol; 45:106-111). This example also found that the use of concentration in a logistic regression mode was the least significant predictor of the three cfDNA parameters tested for timepoint A, but became quite significant and predictive for timepoint B. The low n of this example precluded delineating early PE patients from late PE for the model, but preliminary data suggests concentration may be of significant interest in future model development.

Concentration dynamics between timepoint A and B warrants further exploration and understanding, as it may correlate with PE onset and would further inform about optimal timing for cfDNA screening. In this example fetal fraction was significantly lower for the late-stage PE patients at timepoint A and significantly lower for both the early-stage and late-stage PE patients at timepoint B, compared to the patients that did not develop PE. Other studies have also shown that low FF is associated with PE (Becking et al., 2021, Prenat Diag; 41(10):1296-1304; Gerson et al., 2019, Pregnancy Hypertens; 16:148-153; Yuan et al., 2020, Pregnancy Hypertens; 22:101-108; and Sapantzoglou et al., 2021, J Matern Fetal Neonat Med; 35(25):5363-5368). Rolnik et al. found a significant association between FF result and first trimester markers for adverse pregnancy outcomes including PAPP-A and PlGF, with their results suggesting that patients at increased risk for PE tend to have lower fetal fractions (Rolnik et al., 2018, Ultrasound Obstet Gynecol; 52:722-727).

A lot of research has been carried out to enable easy and effective first trimester screening of patients that will develop PE during their pregnancy. However, to date, no effective biomarker has been identified that would allow for this on a unique blood draw, which may help explain in part why screening for PE has not become universal throughout the world. One of the main strengths of this study is that it was able to develop and test a novel PE screening model that integrated multiple cfDNA signals. The proposed cfDNA model appears to have improved sensitivity compared to the Competing Risk model, which is considered to be the gold standard of screening for PE and could therefore be more effective. In addition, this is the first time that cfDNA fragment size data are reported in patients who will or will not develop PE. Another strength of this cfDNA model is that it is easy to use and so avoids a lot of the complications and special skills needed for other PE screening methods. It would also, in theory, be easy to incorporate into routine noninvasive pre-natal screening. Future iterations of the model will likely expand the model to delineate early vs. late preeclampsia, in addition to including other key variables.

As shown by the data from timepoint A, results could be available before 16 weeks of gestation which would allow for implementation of preventative treatment with aspirin as recommended by ACOG (ACOG Practice Bulletin, Number 222. Gestational Hypertension and Preeclampsia. Obstetrics and gynecology 2020, 135, e237-e260, doi:10.1097/aog.0000000000003891). However, a limitation of this example is the small sample size, with only 12 PE patients in our test population. Future prospective studies would need to be carried out in a much larger cohort of pregnant patients to determine the feasibility of our proposed model in identifying at risk patients.

In conclusion, logistic regression modelling of preeclampsia outcomes with cfDNA fetal fraction, fragment size distribution, and concentration can assist with the probability prediction of pregnancies at risk for development preeclampsia. The proposed model is theoretically a useful additional tool for screening, and subsequently counselling patients about risk and prophylaxis regarding the development of preeclampsia.

This example has now published as Gekas et al., “Use of cell-free signals as biomarkers for early and easy prediction of preeclampsia,” Frontiers in Medicine, 2023 May 24:10:1191163. doi: 10.3389/fmed.2023.1191163. eCollection 2023, which is herein incorporated by reference in its entirety.

### Example 2

### Further Analysis Use of Cell-Free DNA Signals as Biomarkers for Detection of Preeclampsia

A further analysis was undertaken of the data of Example 1 using the following formula:

$${y}_{i} = β0 - β1 ( FragSizeDist ) + {β}_{2} ( FragSizeDist : FF ) - {β}_{3} ( FragSizeDist : FF : Conc ) .$$

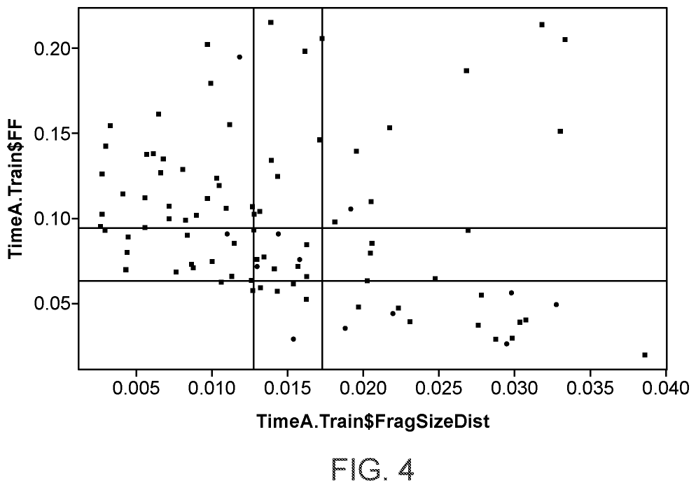

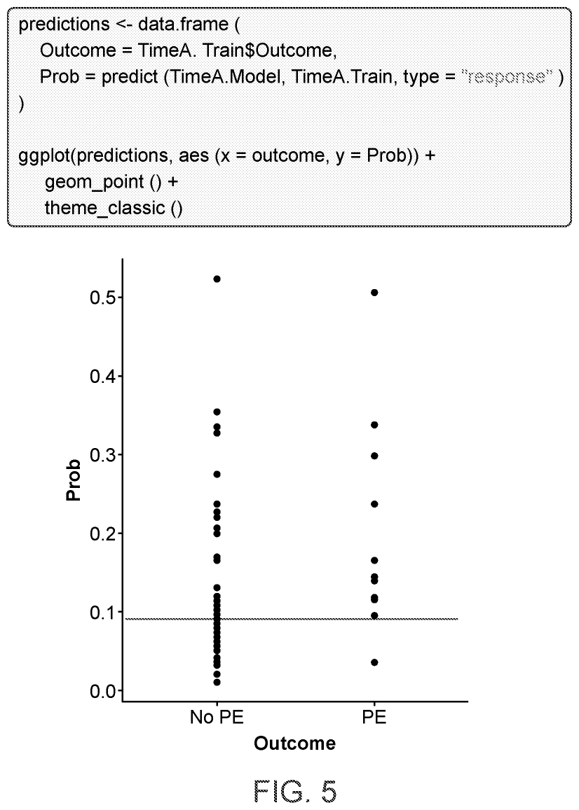

This formula continues to determine a separation between PE and non-PE samples, with a final sensitivity of 83% and a specificity of 62%. FIG. 4 is a linear regression plot showing the use of fetal fraction and fragment size distribution to distinguish between PE samples and control samples at timepoint A. PE samples are denoted by circles. Control samples are denoted by squares. FIG. 5 is a plot of probability versus outcome; Cutoff=0.098; Accuracy: 0.6526; Sensitivity: 0.8333; and Specificity 0.6265.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials, and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of detecting preeclampsia and/or determining an increased risk for preeclampsia in a pregnant female subject, the method comprising:
providing cell free DNA (cfDNA) sequence information obtained from a biosample obtained from the pregnant female subject; and
from the cfDNA sequence information:
determining cell free DNA (cfDNA) concentration;
determining fetal fraction within the cfDNA; and/or
determining fragment size distribution within the cfDNA;
wherein:
a higher cfDNA concentration relative to a normal control;
a lower fetal fraction within the cfDNA relative to a normal control;
a higher fragment size distribution relative to a normal control; and/or
the ratio of fetal fraction to fragment size distribution is less than this ratio in a normal control
is indicative of preeclampsia and/or an increased risk for preeclampsia in the pregnant female.

2. The method of claim 1, w herein the cfDNA sequence information is obtained from sequencing based non-invasive prenatal testing (NIPT) testing.

3. A method of detecting preeclampsia and/or determining an increased risk for preeclampsia in a pregnant female subject, the method comprising:
removing intact cells from a biosample obtained from the pregnant female;
isolating cell free DNA (cfDNA) molecules from the biosample;
sequencing the resulting enriched cfDNA to obtain cfDNA sequence information; and
from the cfDNA sequence information:
determining cell free DNA (cfDNA) concentration;
determining fetal fraction within the cfDNA; and/or
determining fragment size distribution within the cfDNA;
wherein:
a higher cfDNA concentration relative to a normal control;
a lower fetal fraction within the cfDNA relative to a normal control;
a higher fragment size distribution relative to a normal control; and/or
the ratio of fetal fraction to fragment size distribution is less than this ratio in a normal control
is indicative of preeclampsia and/or an increased risk for preeclampsia in the pregnant female.

4. The method of claim 1, comprising:
determining fetal fraction within the cfDNA; and
determining fragment size distribution within the cfDNA;
wherein:
a lower fetal fraction within the cfDNA relative to a normal control; and
a higher fragment size distribution relative to a normal control;
is indicative of preeclampsia and/or an increased risk for preeclampsia in the pregnant female.

5. The method of claim 1, comprising:
determining cell free DNA (cfDNA) concentration;
determining fetal fraction within the cfDNA; and
determining fragment size distribution within the cfDNA;
wherein:
a higher cfDNA concentration relative to a normal control;
a lower fetal fraction within the cfDNA relative to a normal control; and
a higher fragment size distribution relative to a normal control;
is indicative of preeclampsia and/or an increased risk for preeclampsia in the pregnant female.

6. A method comprising:
a) accessing sequence reads from cfDNA and determining:
i) cfDNA concentration;
ii) fetal fraction within the cfDNA;
iii) fragment size distribution within the cfDNA; and/or
iv) a parameter comprising the fetal fraction and the fragment size distribution;
b) comparing
i) the cfDNA concentration to a first threshold amount;
ii) the fetal fraction to a second threshold amount;
iii) the fragment size distribution to a third threshold amount; and/or
iv) the parameter comprising the fetal fraction and the fragment size distribution to a fourth threshold amount;
wherein the first, second, third and fourth threshold amounts are determined based on a plurality of normal samples;
c) determining that the pregnant female subject has preeclampsia and/or an increased risk for preeclampsia, wherein
i) the cfDNA concentration is higher than the first threshold amount;
ii) the fetal fraction is lower than the second threshold amount;
iii) the fragment size distribution is higher than the third threshold amount; and/or
iv) the parameter comprising the fetal fraction and the fragment size distribution is less than the fourth threshold amount;
is indicative of preeclampsia and/or an increased risk for preeclampsia in the pregnant female.

7. The method of claim 1, wherein the biosample is obtained from the pregnant female at less than 16 weeks gestation.

8. The method of claim 1, wherein the biosample is obtained from the pregnant female subject at about 11 to about 14.2 weeks gestation.

9. The method of claim 1, wherein the biosample is obtained from the pregnant female at greater than 20 weeks gestation.

10. The method of claim 1, wherein the biosample is obtained from the pregnant female subject at about 17.6 to about 25.5 weeks gestation.

11. The method of claim 1, wherein detecting preeclampsia and/or determining an increased risk for preeclampsia comprises detecting early-onset preeclampsia and/or determining an increased risk for early-onset preeclampsia.

12. The method of claim 1, wherein detecting preeclampsia and/or determining an increased risk for preeclampsia comprises detecting late onset preeclampsia and/or determining an increased risk for late onset preeclampsia.

13. The method of claim 1, wherein the biosample comprises whole blood, serum, or plasma.

14. The method of claim 1, further comprising providing the pregnant female with a therapeutic intervention selected from the group consisting of increased frequency of prenatal visits, antihypertensive medications to lower blood pressure, corticosteroid medications, anticonvulsant medications, bed rest, hospitalization, early delivery, and combinations thereof, and/or treating the pregnant female with a low dose of aspirin, wherein a low dose of aspirin comprises about 50 to about 150 mg per day.

15. A system comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform the method of claim 1.

16. A machine comprising one or more microprocessors and memory, which memory comprises instructions executable by the one or more microprocessors and which memory comprises sequence reads mapped to a reference genome, wherein the sequence reads are reads of cfDNA from a test sample from a pregnant female subject, and wherein the instructions executable by the one or more microprocessors are configured to perform the method of claim 1.

17. A non-transitory computer-readable storage medium with an executable program stored thereon, where the program instructs a microprocessor to access sequence reads mapped to a reference genome and perform the method of claim 1.
